(19) United States
(12) Patent Application Publication
(10) Pub. No.: US 2014/0107213 A1
(43) Pub. Date: Apr. 17, 2014

(54) METHODS FOR ADMINISTRATION OF COLCHICINE WITH GRAPEFRUIT JUICE

(71) Applicant: Matthew W. Davis, Erwinna, PA (US)

(72) Inventor: Matthew W. Davis, Erwinna, PA (US)

(73) Assignee: TAKEDA PHARMACEUTICALS U.S.A., INC., DEERFIELD, IL (US)

(21) Appl. No.: 14/053,780

(22) Filed: Oct. 15, 2013

Related U.S. Application Data
(63) Continuation of application No. 12/858,706, filed on Aug. 18, 2010, now abandoned.
(60) Provisional application No. 61/235,478, filed on Aug. 20, 2009.

(51) Int. Cl. A61K 31/165; A23L 2/02

(52) U.S. Cl. CPC A61K 31/165; A23L 2/02
USPC 514/629

(57) ABSTRACT

Methods for safe administration of colchicine together with consumption of grapefruit juice are disclosed.

### CROSS-REFERENCE TO RELATED APPLICATION

This application is a continuation of U.S. application Ser. No. 12/858,706, filed Aug. 18, 2010, which claims priority to U.S. Provisional Application No. 61/235,478, filed Aug. 20, 2009; the disclosure of each application is incorporated by reference herein in its entirety.

### BACKGROUND

This disclosure relates to methods allowing for the administration of colchicine for therapeutic purposes together with grapefruit juice with improved safety compared to prior methods of administration.

Colchicine, chemical name (−)-N-[(7S,12aS)-1,2,3,10-tetramethoxy-9-oxo-5,6,7,9-tetrahydrobenzo[a]heptalen-7-yl]-acetamide, is an alkaloid found in extracts of Colchicum autumnale, Gloriosa superba, and other plants. It is a microtubule-disrupting agent used in the treatment of gout and other conditions that may be treated, relieved or prevented with anti-inflammatory treatment. Colchicine impairs the motility of granulocytes and can prevent the inflammatory phenomena that initiate an attack (or flare) of gout. Colchicine also inhibits mitosis, resulting in effects in cells with high turnover rates such as those in the gastrointestinal tract and bone marrow. The primary adverse side effects of colchicine therapy include gastrointestinal upset such as diarrhea and nausea.

Gout (or gouty arthritis) is a disease caused by a build up of uric acid in the joints. Such a build up is typically due to an overproduction of uric acid, or to a reduced ability of the kidney to excrete uric acid. Gout is characterized by excruciating, sudden, unexpected, burning pain, as well as by swelling, redness, warmness, and stiffness in the affected joint. Low-grade fever may also be present. A gout flare is a sudden attack of pain in affected joints, especially in the lower extremities, and most commonly in the big toe. In afflicted individuals, the frequency of gout flares typically increases over time. In this manner, gout progresses from acute gout to chronic gout, which involves repeated episodes of joint pain.

Colchicine can reduce pain in attacks of acute gout flares and also can be used beneficially for treating adults for prophylaxis of gout flares. Although its exact mode of action in the relief of gout is not completely understood, colchicine is known to decrease the inflammatory response to urate crystal deposition by inhibiting migration of leukocytes, to interfere with urate deposition by decreasing lactic acid production by leukocytes, to interfere with kinin formation and to diminish phagocytosis and subsequent inflammatory responses.

Colchicine is also used in treatment of familial Mediterranean fever (FMF), the most common of the autoinflammatory syndromes, which is characterized by recurrent inflammatory attacks of fever and serositis. FMF is an inherited disorder usually occurring in people of Mediterranean origin, including Sephardic Jews, Arabs, Armenians and Turks. The first episode usually occurs in childhood or the teenage years. Typically, attacks last 12 to 72 hours and can vary in severity. The length of time between attacks is also variable. Without treatment to help prevent attacks and complications, a buildup of amyloid depositions (amyloidosis) in the body's organs and tissues may occur, which can lead to organ failure, e.g., kidney failure.

Daily colchicine is the mainstay of therapy for FMF, resulting in complete remission or marked reduction in the frequency, duration, and severity of attacks in most patients. In an appropriate dose it prevents amyloidosis, even if it fails to improve attacks. Colchicine is also effective in arresting and reversing renal amyloidosis. The adult dosage of colchicine is typically 1-3 mg a day, depending on the patient's response, but daily dosages of up to 3 mg are occasionally given to patients, specifically 1.2-2.4 mg/day. In children, the dose of colchicine is adjusted according to their body weight or body surface area. The minimal dose is about 0.25 mg daily in children 1-2 years old. By age 6-7, children can be treated with a dose of 1.0 mg daily. Herein, a child means an individual less than about 18 years old.

Additionally, for individuals with Behçet's syndrome (or disease), colchicine at doses of 1-2.4 mg daily, adjusted to bodyweight, reduced the frequency of genital ulcers, erythema nodosum, and arthritis among women and reduced the occurrence of arthritis among men.

Behçet's disease is an autoimmune disease that results from damage to blood vessels throughout the body, particularly veins. Behçet's disease is common in the Middle East, Asia, and Japan. Behçet's disease tends to develop in people in their 20's or 30's, but people of all ages can develop this disease. Although Behçet's disease affects each person differently, symptoms of Behçet's disease include recurrent ulcers in the mouth (resembling canker sores) and on the genitals, and eye inflammation. The disorder may also cause various types of skin lesions, arthritis, bowel inflammation, meningitis (inflammation of the membranes of the brain and spinal cord), and cranial nerve palsies. Behçet's is a multi-system disease; it may involve all organs and affect the central nervous system, causing memory loss and impaired speech, balance, and movement. Treatment for Behçet's disease is symptomatic and supportive. For disease that is confined to mucocutaneous regions (mouth, genitals, and skin), topical steroids and non-immunosuppressive medications such as colchicine may be effective. In individuals with Behçet's disease, colchicine at doses of 1-2 mg daily, adjusted to bodyweight, reduced the frequency of genital ulcers, erythema nodosum, and arthritis among women and reduced the occurrence of arthritis among men.

Colchicine has a narrow therapeutic index. The margin between an effective dose and a toxic dose of colchicine is much narrower than that of many other widely used active agents. Consequently, actions that result in increased colchicine levels in patients receiving colchicine therapy are particularly dangerous. Co-administration of colchicine to patients along with certain other active agents or foods can have the effect of increasing colchicine levels. Such active agent interactions with colchicine have been reported to result in serious morbid complications and, in some cases, death.

Colchicine is rapidly absorbed from the gastrointestinal tract. Peak concentrations occur in 0.5 to 2 hours. The drug and its metabolites are distributed in leukocytes, kidneys, liver, spleen and the intestinal tract. Colchicine is metabolized in the liver and excreted primarily in the feces with 10 to 20% eliminated unchanged in the urine.

Many cytochrome p450 (CYP) enzymes metabolize active agents in humans. Cytochrome p450 (CYP) enzymes are found in the liver, the gastrointestinal tract and other locations in the body. CYP enzymes occur as a variety of closely related proteins referred to as isozymes and different CYP isozymes may preferentially metabolize different active agents. Biotransformation of colchicine in human liver microsomes to 3-demethylchochicine and 2-demethylcolchicine is correlated with activity of cytochrome p450 isozyme 3A4 (CYP3A4), as shown by experiments using antibodies against CYP3A4 and experiments using chemical inhibition of CYP3A4.

Active agent interactions associated with altered metabolism of an active agent by cytochrome p450 isozymes generally result from alteration of the expression or activity of the CYP by a second active agent. The 3A family of CYP isozymes, particularly CYP3A4, is known to be involved in many clinically significant active agent interactions, including those involving colchicine.

P-glycoprotein (P-gp) is an ATP-dependent cell surface transporter molecule that acts as an ATPase efflux pump, actively pumping certain compounds, including colchicine, out of cells. P-gp is encoded by the adenosine triphosphate-binding cassette subfamily B member 1 (ABCB1) gene, also referred to as the multiple drug resistance 1 gene (MDR1), and is expressed in the human small intestine.

Since colchicine acts intracellularly, the combined effects of inhibition of colchicine metabolism by CYP3A4 and inhibition of P-gp-mediated efflux of colchicine from cells by substances, such as second active agents or chemicals in foods, can cause colchicine toxicity in patients taking what would be a safe dose of colchicine in the absence of concomitant administration of the second active agent or consumption of an inhibiting food.

Active agent interactions present a health risk to patients and a medical challenge for all medical care workers. Various studies of adverse reactions from exposure to active agents have found that 6.5-23% of the adverse reactions result from active agent interactions. Unfortunately, each year a number of deaths occur as the direct result of patients taking a new prescription pharmaceutical product in combination with their existing medication regimen.

For example, a near fatal case of acute colchicine intoxication in a child with familial Mediterranean fever (FMF) was reported. (Goldbart et al. (2000) Eur. J. Pediatr 159:895-897) The child was receiving prophylactic doses of colchicine (2 mg/day) and had been drinking about 1000 mL/day of natural grapefruit juice in the two months prior to the near fatal incident. Goldbart et al. speculated that the combination of long-term high dose colchicine with long-term high grapefruit juice consumption contributed to the colchicine intoxication.

Grapefruit juice can markedly increase oral active agent bioavailability. For example, a single glass (200-250 mL) of grapefruit juice can produce a several-fold increase in the AUC and Cₘₐₓ of orally administered felodipine.

Grapefruit juice inhibits CYP3A4. The grapefruit contains furanocoumarins. Bergamottin and 6′,7′-dihydroxybergamottin are thought to be the two main furanocoumarins responsible for the decrease in CYP3A4 enzyme activity caused by grapefruit juice. When co-administration of grapefruit juice with active agents that are CYP3A4 substrates has caused increases in oral bioavailability, such as for felodipine, there have been no observed delays in the elimination half-life of the active agent. Therefore, the effect of grapefruit juice on active agent metabolism is thought to be primarily due to inhibition of intestinal CY3A4 and not hepatic CYP3A4. Results from several studies have demonstrated that grapefruit juice decreases intestinal CYP3A4 levels (Malhotra et al. (2001) rest of citation)

Further, grapefruit juice has also been shown to inhibit the efflux pump, P-gp. By inhibiting either or both of CYP3A4 and the P-gp enzyme systems, grapefruit juice can cause active agent interactions.

Recently, an in vitro study showed that grapefruit juice effects on P-gp augmented colchicine intestinal absorption, suggesting that grapefruit juice is the source of a potentially hazardous interaction with colchicine, resulting in augmenting colchicine oral bioavailability and severely toxic side effects. (Dahon & Amidon (2009) Pharm. Res. 26(4):883-892)

There accordingly remains a need for improved methods for administering colchicine to individuals who are concomitantly drinking grapefruit juice so as to reduce the possibility of colchicine toxicity. The present disclosure addresses this need and provides further advantages.

### SUMMARY

Disclosed herein are methods for administering colchicine to individuals who are consuming grapefruit juice or grapefruits so as to reduce the possibility of colchicine toxicity.

In an embodiment, a method of administering colchicine to a patient in need thereof comprises administering a daily dosage amount of colchicine orally to a patient consuming a daily amount of grapefruit juice that does not exceed 16 fluid ounces.

### DETAILED DESCRIPTION

Disclosed herein are methods for safely administering colchicine with grapefruit juice, a CYP3A4 inhibitor and a P-gp inhibitor. It has now been discovered that oral administration of colchicine with consumption of a recommended maximum daily amount of grapefruit juice is bioequivalent to administration of colchicine in the absence of grapefruit juice consumption. Thus, contrary to previous belief, it is disclosed herein that colchicine can be administered safely to a patient with consumption of grapefruit juice in amounts no greater than about 16 fluid ounces (about 480 mL) daily. Consumption of 8 fluid ounces of grapefruit juice is equivalent to consumption of a whole medium grapefruit. A medium grapefruit has a diameter of about 4 inches and a mass of about 250 grams. (See, for example, grapefruit and grapefruit juice entries in the United States Department of Agriculture National Nutrient Database.) Herein, consumption of grapefruit juice in an amount no greater than 16 fluid ounces daily includes consumption of no more than two whole medium grapefruit daily, or daily consumption of any combination of grapefruit juice and grapefruit equivalent to 16 fluid ounces of grapefruit juice, e.g., one whole medium grapefruit and 8 fluid ounces of grapefruit juice.

Colchicine can be used to treat or prevent various diseases or conditions, including, for example, an acute gout flare, chronic gout, acute pericarditis, asthma, Behçet's disease, cancer, a pseudogout cystic disease comprising polycystic kidney disease or cystic fibrosis, a demyelinating disease of central or peripheral origin, Dupuytren's contracture, familial Mediterranean fever, glaucoma, idiopathic pulmonary fibrosis, idiopathic thrombocytopenic purpura, an inflammatory disorder comprising rheumatoid arthritis, lentiviral infection, multiple sclerosis, postpericardiotomy syndrome, primary amyloidosis, primary biliary cirrhosis, proliferative vitreoretinopathy, pyoderma gangrenosum, recurrent pericarditis, or a need for enhanced bone formation or bone mineral density.

In the specification and claims that follow, references will be made to a number of terms which shall be defined to have the following meaning.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism.

“Active agent interaction” refers to a change in the metabolism or the pharmacology of an active agent in a patient that can occur with co-administration of a second active agent. A “potential active agent interaction” refers to an active agent interaction between two active agents that is theoretically possible based on knowledge that one of the active agents is metabolized by a given cytochrome p450 isozyme or transported by P-gp and that the second of the active agents is a substrate, inhibitor, or inducer of that cytochrome p450 isozyme or an inhibitor or inducer of P-gp.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

“Bioequivalence” or “equivalent bioavailability” means the absence of a significant difference in the rate or extent to which the active agent in pharmaceutical equivalents or pharmaceutical alternatives is absorbed into a living system or is made available at the site of physiological activity or the absence of a significant difference in the rate or extent to which the active agent in a pharmaceutical composition is absorbed into a living system or is made available at the site of physiological activity when administered by two different methods (e.g., dosing under non-fasted versus fasted conditions). Bioequivalence can be determined by comparing in vitro dissolution testing data for two dosage forms or two dosing conditions or by comparing pharmacokinetic parameters for two dosage forms or two dosing conditions.

In some embodiments, two pharmaceutical products or two methods (e.g., dosing under fed versus fasted conditions) are bioequivalent if the ratio of the geometric mean of logarithmic transformed AUC_{0-∞}, AUC₀₋ₜ, or Cₘₐₓ for the two products or two methods is about 0.80 to about 1.25; specifically if the 90% Confidence Interval (CI) upper and lower limits for the ratio of the geometric mean of logarithmic transformed AUC_{0-∞}, AUC₀₋ₜ, or Cₘₐₓ for the two products or two methods are each about 0.80 to about 1.25; more specifically if the ratios of the geometric mean of logarithmic transformed AUC_{0-∞}, AUC₀₋ₜ, and Cₘₐₓ for the two products or two methods are about 0.80 to about 1.25; yet more specifically if the 90% Confidence Interval (CI) upper and lower limits for the ratios of the geometric mean of logarithmic transformed AUC_{0-∞}, AUC₀₋ₜ, and Cₘₐₓ for the two products or two methods are each about 0.80 to about 1.25.

“Colchicine therapy” refers to medical treatment of a symptom, disorder, or condition by administration of colchicine. Colchicine therapy can be considered optimal when effective plasma levels are reached when required.

A “dose” means the measured quantity of a drug to be taken at one time by a patient.

A “dosage amount” means an amount of a drug suitable to be taken during a fixed period, usually during one day (i.e. daily). A “daily dosage amount” is the total dosage amount taken in one day, that is, a 24-hour period.

The term “effective amount” or “therapeutically effective amount” means an amount effective, when administered to a patient, to provide any therapeutic benefit. A therapeutic benefit may be an amelioration of symptoms, e.g., an amount effective to decrease the symptoms of acute gouty arthritis, for example pain associated with an attack of acute gouty arthritis. The amount that is “effective” will vary from subject to subject, depending on the age and general condition of the individual, the particular active agent, and the like. Thus, it is not always possible to specify an exact “effective amount.” However, an appropriate “effective” amount in any individual case may be determined by one of ordinary skill in the art using routine experimentation. In certain circumstances a patient may not present symptoms of a condition for which the patient is being treated. A therapeutically effective amount of an active agent may also be an amount sufficient to provide a significant positive effect on any indicium of a disease, disorder, or condition, e.g. an amount sufficient to significantly reduce the severity of an attack of acute gouty arthritis. A significant effect on an indicium of a disease, disorder, or condition is statistically significant in a standard parametric test of statistical significance, for example Student's T-test, where p≦0.05. An “effective amount” or “therapeutically effective amount” of colchicine may also be an amount of about 10 mg per day or less, specifically about 8 mg per day or less, or of any dosage amount approved by a governmental authority such as the United States Food and Drug Administration (FDA), for use in treatment. For example, an effective amount can be up to 1.8 mg colchicine per incident of acute gout, or 0.5 or 0.6 mg colchicine twice daily for either prophylaxis of chronic gout or treatment of Behçet's disease or Familial Mediterranean fever. In some embodiments amounts of up to 8 mg colchicine per day, 1.0 or 1.2 mg colchicine per unit dosage form, or 0.5 or 0.6 mg colchicine or less per unit dosage form is an “effective amount” or “therapeutically effective amount” of colchicine.

“Efficacy” means the ability of an active agent administered to a patient to produce a therapeutic effect in the patient.

A “patient” means a human or non-human animal in need of medical treatment. Medical treatment can include treatment of an existing condition, such as a disease or disorder, prophylactic or preventative treatment, or diagnostic treatment. In preferred embodiments the patient is human.

“Risk” means the probability or chance of adverse reaction, injury, or other undesirable outcome arising from a medical treatment. An “acceptable risk” means a measure of the risk of harm, injury, or disease arising from a medical treatment that will be tolerated by an individual or group. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small, or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At risk” means in a state or condition marked by a high level of risk or susceptibility.

Pharmacokinetic parameters referred to herein describe the in vivo characteristics of drug (or a metabolite or a surrogate marker for the drug) over time. These include plasma concentration (C), as well as Cₘₐₓ, Cₙ, C₂₄, Tₘₐₓ, and AUC. “Cₘₐₓ” is the measured plasma concentration of the active agent at the point of maximum, or peak, concentration. “Cₘᵢₙ” is the measured plasma concentration of the active agent at the point of minimum concentration. “Cₙ” is the measured plasma concentration of the active agent at about n hours after administration. “C₂₄” is the measured plasma concentration of the active agent at about 24 hours after administration. The term “Tₘₐₓ” refers to the time from drug administration until Cmax is reached. “AUC” is the area under the curve of a graph of the measured plasma concentration of an active agent vs. time, measured from one time point to another time point. For example AUC₀₋ₜ is the area under the curve of plasma concentration versus time from time 0 to time t, where time 0 is the time of initial administration of the drug. Time t can be the last time point with measurable plasma concentration for an individual formulation. The AUC_{0-∞}, AUC_{0-∞} or AUC_{0-inf} is the calculated area under the curve of plasma concentration versus time from time 0 to time infinity. In steady-state studies, AUC_{O-τ} is the area under the curve of plasma concentration over the dosing interval (i.e., from time 0 to time τ (tau), where tau is the length of the dosing interval. Other pharmacokinetic parameters are the parameter Kₑ or Kₑₗ, the terminal elimination rate constant calculated from a semi-log plot of the plasma concentration versus time curve; t_{1/2} the terminal elimination half-life, calculated as 0.693/Kₑₗ. CL/F denotes the apparent total body clearance after administration, calculated as Total Dose/Total AUC_{∞}; and Vₐᵣₑₐ/F denotes the apparent total volume of distribution after administration, calculated as Total Dose/(Total AUC_{∞}×Kₑₗ).

“Safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

The terms “treating” and “treatment” mean implementation of therapy with the intention of reducing in severity or frequency symptoms, elimination of symptoms or underlying cause, prevention of the occurrence of symptoms or their underlying cause, and improvement or remediation of damage.

“Side effect” means a secondary effect resulting from taking a drug. The secondary effect can be a negative (unfavorable) effect (i.e., an adverse side effect) or a positive (favorable) effect.

The most frequently reported adverse side effects to colchicine therapy are gastrointestinal, specifically abdominal pain with cramps, diarrhea, nausea, and vomiting. Less frequently or rarely reported adverse side effects associated with colchicine therapy include anorexia, agranulocytosis, allergic dermatitis, allergic reactions, alopecia, angioedema, aplastic anemia, bone marrow depression, myopathy, neuropathy, skin rash, thrombocytopenic disorder, and urticaria.

Whether a patient experiences an adverse side effect can be determined by obtaining information from the patient regarding onset of certain symptoms which may be indicative of the adverse side effect, results of diagnostic tests indicative of the adverse side effect, and the like.

Enzymes involved in Phase I and Phase II active agent metabolism, such as the cytochrome p450 isozymes, respond to the constantly changing types and amounts of substrates they encounter. For example, changes in active agent metabolism due to competition for the same cytochrome P450 isozyme can change the clinical effectiveness or safety of an active agent by altering the plasma concentration of the active agent or its metabolite(s). Similarly, inhibition or induction of the cytochrome P450 isozyme that metabolizes a particular active agent can change the clinical effectiveness or safety of that active agent.

For the case in which the active agent is a narrow therapeutic index active agent, such as colchicine, too little of the active agent in the blood stream can lead to insufficient therapeutic activity, while too much of the active agent in the blood stream can lead to excessive therapeutic activity or toxicity, either of which can be detrimental to the patient. Therefore, since colchicine is a substrate of CYP3A4, the administration of colchicine with a substance that is a substrate, inhibitor, or inducer of CYP3A4 can affect the metabolism of colchicine by that cytochrome P450. Administration of a substrate or an inhibitor of CYP3A4 with colchicine can decrease the metabolism of colchicine by CYP3A4, resulting in higher colchicine plasma concentrations and potential toxicity, while administration of an inducer of CYP3A4 with colchicine can increase the metabolism of colchicine by CYP3A4, resulting in lower colchicine plasma concentrations and reduced efficacy.

Similarly, inhibition or induction of the P-glycoprotein (P-gp) transporter that actively pumps a particular active agent out of cells can change the clinical effectiveness or safety of that active agent.

In an embodiment, a method of treating a patient with colchicine comprises administering a dosage amount of colchicine and an amount of grapefruit juice daily to a patient in need of colchicine therapy, wherein the amount of grapefruit juice does not exceed 16 fluid ounces. The patient in need of colchicine therapy can be, for example, a patient with chronic gout needing treatment to prevent gout flares, a patient needing treatment of an acute gout flare, a patient needing treatment of FMF or Behçet's disease.

To treat an acute gout flare the dose of colchicine can be about 1.0 to about 1.2 mg of colchicine, for example, two tablets each comprising about 0.6 mg colchicine, upon onset of the flare, followed by another dose of 0.5 or 0.6 mg colchicine in about an hour. The dose to treat an acute gout flare can also be 1.0 to about 1.2 mg of colchicine upon onset of the flare, followed by a 0.5 mg or 0.6 mg dose every hour, or two 0.5 mg or 0.6 mg doses every two hours, until pain is relieved or until diarrhea ensues (“diarrheal dose”). Various other dosing regimens for an acute gout flare have been used in the art. For chronic gout, a prophylactic dose of colchicine to prevent acute gout flares can be about 0.5 or 0.6 mg once or twice daily.

To treat familial Mediterranean fever the dose of colchicine can be a total of about 0.3 to about 2.4 mg colchicine daily. To treat Behçet's disease the dose of colchicine can be about 0.5 or 0.6 mg colchicine twice daily.

The method can also comprise monitoring a patient, for example monitoring the patient for an adverse reaction, a side effect, or a symptom of an active agent interaction or monitoring the patient's plasma concentration of colchicine. The method can also comprise adjusting administration or dosing of colchicine for the patient or the volume of grapefruit juice consumed by the patient. The adjustment can be based on the monitoring, for example based on the determined plasma concentration of colchicine. Adjusting administration of colchicine to the patient to avoid an adverse side effect or adjusting dosing regimens can be performed by one of ordinary skill in the art, taking into consideration the physiology of the patient, including such factors as the age, sex, and health of the patient, as well as active agents the patient may be taking at the time. Optionally, the patient can be monitored at the initial, or a subsequent, stage of treatment to ensure therapeutic plasma levels of colchicine are achieved or maintained.

Monitoring the patient can comprise monitoring the patient's plasma concentration of colchicine; monitoring the patient for symptoms of an active agent interaction between colchicine and grapefruit juice; monitoring the patient for an adverse reaction (e.g., toxicity) resulting from administration of colchicine with the grapefruit juice; monitoring the patient for an adverse reaction (e.g., toxicity) associated with colchicine; monitoring the patient for an adverse reaction associated with increased plasma concentration of colchicine; or monitoring the patient for a symptom of increased plasma concentration of colchicine.

Monitoring the patient can be monitoring any appropriate patient-specific, disease-specific, or substance-specific parameter appropriate to avoid or safely manage an active agent interaction. Monitoring the patient can be, for example, monitoring the patient for an adverse reaction, a side effect, or a symptom of an active agent interaction, for example by physical examination or visual identification; monitoring the blood level of colchicine in the patient; monitoring clinical laboratory tests appropriate for colchicine or a medical diagnosis for the patient; monitoring therapeutic effect of colchicine on the patient's condition; monitoring occurrence in the patient of a known side effect, sub-therapeutic outcome, or adverse reaction of colchicine; monitoring the patient for occurrence of an unexpected response during treatment; monitoring changes in control, signs, or symptoms of a condition of the patient, or determining a complete list of medical diagnoses for the patient. To monitor a patient for an adverse reaction, sub-therapeutic outcome, or a side effect, a doctor may order blood or urine tests, including but not limited to blood samples for white blood count, liver function tests, blood chemistries, EKGs, blood urea nitrogen and creatinine. Any other tests known to those skilled in the art that would help determine or assess the presence of and/or extent or significance of an adverse event, adverse reaction, or side effect. Monitoring the patient can be performed by the patient or by a medical care worker.

In all of the embodiments herein, a medical care worker can determine the plasma concentration of colchicine by performing or ordering the performance of any suitable method. For example, the medical care worker could order a test using blood drawn from the patient for determining the plasma concentration of colchicine.

The following examples further illustrate aspects of this disclosure but should not be construed as in any way limiting its scope. In particular, the conditions are merely exemplary and can be readily varied by one of ordinary skill in the art.

### Example 1

This study is an open-label, non-randomized, single-center, one-sequence, two-period drug interaction study conducted in healthy male and female volunteers. Twenty-four (24) non-smoking, non-obese adult volunteers will be enrolled. All subjects will be dosed and studied as a single cohort, with each subject receiving the same treatment in a non-randomized fashion. As described below, a single dose of colchicine, 0.6 mg, will be administered alone on Day 1, and then co-administered with the grapefruit juice on Day 18. Grapefruit juice will be administered for 4 consecutive days (1×240 mL twice daily Days 15-17 and Day 18 (PM) alone and co-administered with a single 0.6 mg colchicine tablet in the morning on Day 18 (AM) (1×240 mL) beginning on the morning of Day 15, with the last 240 mL grapefruit juice dose administered in the evening of Day 18. A 14-day washout period will be completed after the first colchicine dose on Day 1 and prior to the administration of the first grapefruit juice dose on Day 15.

Grapefruit juice (THIRSTER® brand) will be purchased and stored according to the labeled instructions. Subjects in the study will consume 240 mL (8 fluid ounces) of juice every 12 hours for 8 doses (4 day regimen) to ensure the maximum inhibitory effect.

Description of Treatments

| Treatment | Description (Source) |
|---|---|
| A | Colchicine Tablets USP, 0.6 mg (Mutual provided by |
| | Sponsor as bulk drug supplies in 100-count, capped |
| | HDPE bottle) |
| B | Grapefruit juice, 240 mL consumed twice daily [(4-day |
| | regimen) to be provided by Cetero Research from |
| | commercial sources] |

Following a screening period of up to 28 days, 24 healthy male and female volunteers will be enrolled. Subjects will be confined to the study unit for approximately 1.5 days on two separate occasions starting at least 10 hours prior to dosing on the evening of Day 1 and Day 17 until 24 hours following study drug administration on Day 1 and Day 18. In addition to confinement, study subjects will return to the clinical study site for the collection of blood samples 36, 48, 72, and 96 hours post dosing during both colchicine pharmacokinetic sampling periods. Total study participation, exclusive of up to 28 days of screening, will be approximately 22 days, during which subjects will be confined on two occasions for a total confinement of approximately 3 days.

All subjects will receive a single 0.6 mg dose of colchicine on Day 1, followed by a 14-day washout period in which subjects will not be confined at study site. At discharge on Day 2 (at approximately 8 AM), study subjects will be instructed to return to the clinical site on Days 15 through 17 in the mornings and evenings to consume (1×240 mL, i.e., 8 fluid ounces) grapefruit juice twice daily on Days 15 through 17 in a ‘directly-observed’ fashion. On the evening of Day 17, study participants will return to the clinic for their final study confinement period. In the morning of Day 18, study subjects will receive a single 0.6 mg colchicine dose with a 240 mL serving of grapefruit juice. In the evening of Day 18, study subjects will receive their final 240 mL serving of grapefruit juice administered alone.

A minimum fasting period of 10 hours (overnight fast) will be in effect before administration of colchicine on Day 1 and prior to the administration of colchicine and grapefruit juice on Day 18. Fasting will continue for 4 hours post-dosing on Days 1 and 18 after the morning administration. Grapefruit juice doses administered on Days 15-17 and Day 18 (PM) will not be in a fasted state. For colchicine, medication must be swallowed whole, not chewed. For grapefruit juice, the entire serving must be consumed. For all treatments, the subjects will not be permitted to lie down for the first 4 hours following administration of drug to ensure proper stomach emptying. On both of the confined study days after the morning administrations (Day 1 and Day 18), water will be allowed ad libitum beginning 2 hours post-dose. Intake of all study medications will be supervised. Standardized meals will be served according to a pre-determined schedule throughout the study.

Serial blood samples will be collected by individual venipuncture up to 96 hours following drug administration on Day 1 and Day 18. Blood samples for determination of colchicine plasma concentrations will be obtained at time zero (pre-dose) and after dose administration at 0.5, 1.0, 1.5, 2, 3, 4, 5, 6, 8, 12, and 24 hours post-dose during the confinement period. Subjects will then return on a non-confined basis to the clinic for continued blood sampling collection at 36, 48, 72, and 96 hours post-dose administration. The furanocoumarins, bergamottin and 6′,7′-dihydroxybergamottin, present in grapefruit juice will not be measured.

Subjects will be monitored regarding adverse events throughout study participation. A complete physical examination and medical history, including measurement of height and weight, will be performed at screening only; a brief targeted physical examination will be conducted at each check-in and prior to study discharge in response to changes in medical history. A single 12-lead electrocardiogram (ECG) will be obtained at screening, but may be repeated at other times as needed in response to adverse events or changes in medical history. A review of all prescribed and over the counter medication(s), including vitamin/supplement use and hormonal contraception, will be obtained at screening, baseline (Period 1) and at check-in for Period 2. Vital signs (seated blood pressure and pulse, respiratory rate and temperature) will be measured at screening, baseline, and upon study discharge. Blood pressure and pulse will also be measured pre-dose and at 1, 2, and 3 hours post-dosing on Days 1 and 18 (AM) to coincide with peak plasma concentrations of colchicine. Clinical laboratory testing, including serum pregnancy tests for women, will be conducted at screening, baseline and prior to discharge from the study. A brief targeted physical examination will be performed, if needed, before discharge.

Thirty-two (32) blood samples will be collected from each subject during the study, for a total blood collection for pharmacokinetic analysis of approximately 192 mL (6 mL per sample×32). Additional blood will be drawn for clinical laboratory evaluations and for pregnancy testing in women.

The non-compartmental pharmacokinetic parameters listed below will be calculated for colchicine using the software package WinNonlin® (Version 5.0.1) designed specifically for the analysis of pharmacokinetic data to calculate the pharmacokinetic parameters. More specifically, WinNonlin®, Model 200 for extravascular input will be utilized.

Protocol-scheduled times will be used unless actual collection times deviate significantly. Deviations will be considered significant if the time between scheduled and actual times is more than the following (see table below).

| | |
|---|---|
| Nominal Time | Reporting Standard |

| | |
|---|---|
| Pre-dose | Within 1 hour prior (to first dose) |

| | | | |
|---|---|---|---|
| 0.0-0.5 | hours | ≦±30 | seconds |
| >0.5-2.0 | hours | ≦±60 | seconds |
| >2.0-8.0 | hours | ≦±2 | minutes |
| >8.0-24 | hours | ≦±5 | minutes |
| >24-48 | hours | ≦±10 | minutes |
| >48 | hours | ≦±1 | hour |

Values for the following pharmacokinetic parameters will be computed for colchicine:
Cₘₐₓ Peak plasma concentration as observed
Tₘₐₓ Time of the peak plasma concentration
AUC₀₋ₜ Area under the plasma concentration versus time curve beginning from the first dose until the last quantifiable concentration, calculated by the linear trapezoidal rule.
AUC_{0-∞} Area under the plasma concentration versus time curve extrapolated to infinity. AUC_{0-∞} is calculated as the sum of AUC₀₋ₜ plus the ratio of the last measurable plasma concentration to the elimination rate constant.
Kₑₗ Apparent first-order terminal elimination rate constant calculated from a semi-log plot of the plasma concentration versus time curve. The parameter will be calculated by linear least-squares regression analysis using the maximum number of points in the terminal log-linear phase (e.g., three or more non-zero plasma concentrations).
Vₐᵣₑₐ/F Apparent total volume of distribution after administration, calculated as Total Dose/(AUC_{0-∞}×Kₑₗ)
CL/F Apparent total body clearance after administration, calculated as Dose/AUC_{0-∞}; weight-adjusted CL/F will also be calculated.
t_{1/2} Apparent first-order terminal elimination half-life will be calculated as 0.693/Kₑₗ.

AUC₀₋ₜ, AUC_{0-∞} and Cₘₐₓ will be log transformed prior to analysis and Tmax will be analyzed without transformation. Ninety percent confidence intervals (90% CI) for the ratio of test (i.e., colchicine administered with grapefruit juice) versus reference (i.e., colchicine administered alone) will be calculated for each parameter, consistent with the two one-sided tests approach. The no drug interaction criterion is a 90% CI for the ratio for AUC₀₋ₜ and Cₘₐₓ of between 0.80 to 1.25, representing a maximum of 20% difference between treatments.

Arithmetic means, coefficients of variation (CV %), standard deviation (SD), median, minimum and maximum values, and number of observations will be calculated for the plasma concentrations and the pharmacokinetic parameters listed above. Geometric mean and geometric coefficient of variation will be provided for AUC₀₋ₜ and Cₘₐₓ.

Analysis of variance (ANOVA) will be performed on the ln-transformed AUC₀₋ₜ, AUC_{0-∞}, and Cₘₐₓ. The ANOVA model will include treatment, sequence, and treatment within a sequence as fixed effects, and subject as a random effect. Each ANOVA will include calculation of least squares mean (LSM), the difference between treatment LSM, and the standard error associated with the difference. These will be done using SAS® GLM procedure.

Ratios of LSM will be calculated using the exponentiation of the difference between treatment LSM from the analyses on the ln-transformed AUC₀₋ₜ, AUC_{0-∞}, and Cₘₐₓ. These ratios will be expressed as a percentage relative to the reference treatment. Consistent with the two one-sided tests for the drug interaction, 90% confidence intervals for the ratios will be derived by exponentiation of the confidence intervals obtained for the difference between treatment LSM resulting from the analyses on the ln-transformed AUC and Cₘₐₓ. The confidence intervals will be expressed as a percentage relative to the reference treatment.

Tₘₐₓ will be analyzed using nonparametric analysis (Walsh Averages and appropriate quantile of the Wilcoxon Signed Rank Test statistic).

Results from a study conducted in general accordance with the above protocol are shown below.

Summary of Statistical Analysis (All Subjects, N = 21)
Ln-Transformed Data

| | | | | | | 90% Confidence | | | |
|---|---|---|---|---|---|---|---|---|---|
| | Least Squares Mean | | Geometric Mean | | | Interval | P-values for | | |
| PK | TRT | TRT | TRT | TRT | | (Lower Limit, | Product | Power of | ANOVA |
| Variable | (A + B) | A | (A + B) | A | % Ratio | Upper Limit) | Effects | ANOVA | % CV |
| Cₘₐₓ | 7.567 | 7.637 | 1934.13 | 2074.12 | 93.25 | (83.07, 104.67) | 0.3094 | 0.6307 | 21.97% |
| AUC₀₋ₜ | 9.030 | 9.083 | 8353.37 | 8806.78 | 94.85 | (86.47, 104.04) | 0.3361 | 0.8170 | 17.51% |
| AUC_{0-∞} | 9.231 | 9.259 | 10212.90 | 10500.20 | 97.26 | (88.08, 107.4) | 0.6347 | 0.7617 | 18.79% |

Geometric means are based on least squares means of ln-transformed values.

Non-Transformed Data

| | | | | 90% Confidence | | |
|---|---|---|---|---|---|---|
| | Least Squares Mean | | | Interval | P-values for | |
| | TRT | TRT | | (Lower Limit, | Product | Power of |
| PK Variable | (A + B) | A | % Ratio | Upper Limit) | Effects | ANOVA |
| Cₘₐₓ | 1971.10 | 2172.96 | 90.71 | (78.8, 102.62) | 0.1936 | 0.5139 |
| AUC₀₋ₜ | 8823.40 | 9329.53 | 94.58 | (83.83, 105.32) | 0.3942 | 0.5991 |
| AUC_{0-∞} | 10854.22 | 11079.98 | 97.96 | (85.84, 110.08) | 0.7748 | 0.5004 |
| Tₘₐₓ | 1.50 | 1.24 | 121.15 | (108.79, 133.52) | 0.0079 | 0.4848 |
| Kel | 0.1527 | 0.1714 | 89.10 | (77.83, 100.36) | 0.1107 | 0.5590 |
| t_{1/2} | 5.58 | 4.64 | 120.25 | (89.32, 151.18) | 0.2722 | 0.1200 |
| Vₐᵣₑₐ/F | 433.06 | 363.89 | 119.01 | (105.19, 132.83) | 0.0278 | 0.4052 |
| CL/F | 62.18 | 60.25 | 103.19 | (95.07, 111.31) | 0.5053 | 0.8326 |
| Weight-Adjusted CL/F | 0.76 | 0.74 | 102.62 | (94.41, 110.84) | 0.5877 | 0.8238 |

P-value of Tmax calculated using Wilcoxon Signed Rank Test is 0.0156

Individual Pharmacokinetic Parameters - Colchicine Alone (All Subjects)

| | | | | | | | | | | | | Weight- | | | |
|---|---|---|---|---|---|---|---|---|---|---|---|---|---|---|---|
| | | | | | | | | | | | | Adjusted | | | |
| | | | | AUC₀₋ₜ | AUC_{0-∞} | | | | | | | CL/F | | | |
| Sub- | Study | Gen- | Treat- | (pg-hr/ | (pg-hr/ | Cₘₐₓ | Tₘₐₓ | Kel | t_{1/2} | Vₐᵣₑₐ/F | CL/F | (L/hr/ | Ln-Transformed | | |
| ject | Day | der | ment | mL) | mL) | (pg/mL) | (hr) | (1/hr) | (hr) | (L) | (L/hr) | kg) | AUC₀₋ₜ | AUC_{0-∞} | Cₘₐₓ |
| 1 | Day 1 | Fe- | A | 8238.50 | 9553.13 | 1930 | 1.50 | 0.1925 | 3.60 | 326.22 | 62.81 | 1.06 | 9.017 | 9.165 | 7.565 |
| | | male | | | | | | | | | | | | | |
| 2 | Day 1 | Male | A | 9269.10 | 10833.60 | 2421 | 1.50 | 0.1619 | 4.28 | 342.07 | 55.38 | 0.56 | 9.134 | 9.290 | 7.792 |
| 3 | Day 1 | Fe- | A | 12892.75 | 14154.74 | 3055 | 1.50 | 0.2194 | 3.16 | 193.19 | 42.39 | 0.57 | 9.464 | 9.558 | 8.025 |
| | | male | | | | | | | | | | | | | |
| 5 | Day 1 | Male | A | 8179.85 | 9548.50 | 1748 | 1.00 | 0.1653 | 4.19 | 380.20 | 62.84 | 0.70 | 9.009 | 9.164 | 7.466 |
| 6 | Day 1 | Male | A | 8056.15 | 10507.75 | 1968 | 1.00 | 0.1160 | 5.98 | 492.22 | 57.10 | 0.62 | 8.994 | 9.260 | 7.585 |
| 7 | Day 1 | Fe- | A | 11316.85 | 13076.36 | 2436 | 1.00 | 0.1881 | 3.68 | 243.91 | 45.88 | 0.68 | 9.334 | 9.479 | 7.798 |
| | | male | | | | | | | | | | | | | |
| 8 | Day 1 | Male | A | 16395.00 | 20677.02 | 3758 | 1.00 | 0.0484 | 14.32 | 599.39 | 29.02 | 0.36 | 9.705 | 9.937 | 8.232 |
| 9 | Day 1 | Male | A | 7594.10 | 9196.04 | 2386 | 1.50 | 0.2291 | 3.03 | 284.79 | 65.25 | 0.83 | 8.935 | 9.127 | 7.777 |
| 10 | Day 1 | Male | A | 5689.30 | 7562.06 | 1315 | 1.00 | 0.1859 | 3.73 | 426.86 | 79.34 | 0.96 | 8.646 | 8.931 | 7.182 |
| 11 | Day 1 | Male | A | 9320.20 | 10953.21 | 1812 | 2.00 | 0.1655 | 4.19 | 331.07 | 54.78 | 0.55 | 9.140 | 9.301 | 7.502 |
| 12 | Day 1 | Male | A | 8835.05 | 10421.12 | 2166 | 1.00 | 0.1563 | 4.43 | 368.37 | 57.58 | 0.57 | 9.086 | 9.252 | 7.681 |
| 13 | Day 1 | Male | A | 10247.65 | 11621.37 | 2060 | 1.00 | 0.1843 | 3.76 | 280.11 | 51.63 | 0.59 | 9.235 | 9.361 | 7.630 |
| 14 | Day 1 | Male | A | 9262.00 | 10409.02 | 2052 | 2.00 | 0.1946 | 3.56 | 296.22 | 57.64 | 0.67 | 9.134 | 9.250 | 7.627 |
| 15 | Day 1 | Fe- | A | 10579.20 | 12505.31 | 2947 | 1.00 | 0.1506 | 4.60 | 318.56 | 47.98 | 0.71 | 9.267 | 9.434 | 7.989 |
| | | male | | | | | | | | | | | | | |
| 16 | Day 1 | Male | A | 6668.65 | 8251.30 | 1451 | 1.00 | 0.1355 | 5.12 | 536.77 | 72.72 | 1.03 | 8.805 | 9.018 | 7.280 |
| 17 | Day 1 | Male | A | 6011.35 | 7559.29 | 1658 | 1.00 | 0.2178 | 3.18 | 364.36 | 79.37 | 1.00 | 8.701 | 8.931 | 7.413 |
| 18 | Day 1 | Fe- | A | 18396.80 | 21667.86 | 2994 | 2.00 | 0.0847 | 8.19 | 327.00 | 27.69 | 0.37 | 9.820 | 9.984 | 8.004 |
| | | male | | | | | | | | | | | | | |
| 19 | Day 1 | Male | A | 8754.96 | 10261.54 | 2284 | 1.00 | 0.1512 | 4.58 | 386.70 | 58.47 | 0.70 | 9.077 | 9.236 | 7.734 |
| 20 | Day 1 | Male | A | 6815.10 | 8576.77 | 2013 | 1.00 | 0.1866 | 3.71 | 374.82 | 69.96 | 0.83 | 8.827 | 9.057 | 7.607 |
| 21 | Day 1 | Male | A | 9564.60 | 10795.16 | 2333 | 1.00 | 0.1851 | 3.74 | 300.24 | 55.58 | 0.52 | 9.166 | 9.287 | 7.755 |
| 22 | Day 1 | Male | A | 3832.87 | 4548.46 | 845.2 | 1.00 | 0.2814 | 2.46 | 468.69 | 131.91 | 1.74 | 8.251 | 8.423 | 6.740 |

| | | | | | | | | | | | | |
|---|---|---|---|---|---|---|---|---|---|---|---|---|
| N | 21 | 21 | 21 | 21 | 21 | 21 | 21 | 21 | 21 | 21 | 21 | 21 |
| Mean | 9329.53 | 11079.98 | 2172.96 | 1.24 | 0.1714 | 4.64 | 363.89 | 60.25 | 0.74 | 9.083 | 9.259 | 7.637 |
| Stdev | 3367.85 | 3949.27 | 652.99 | 0.37 | 0.05 | 2.52 | 97.34 | 21.26 | 0.30 | 0.35 | 0.33 | 0.32 |
| % Cv | 36.10 | 35.64 | 30.05 | 30.27 | 29.22 | 54.29 | 26.75 | 35.29 | 40.60 | 3.83 | 3.60 | 4.25 |
| Median | 8835.05 | 10421.12 | 2060 | 1.00 | 0.1843 | 3.76 | 342.07 | 57.58 | 0.68 | 9.086 | 9.252 | 7.630 |
| Min | 3832.87 | 4548.46 | 845.2 | 1.00 | 0.0484 | 2.46 | 193.19 | 27.69 | 0.36 | 8.251 | 8.423 | 6.740 |
| Max | 18396.80 | 21667.86 | 3758 | 2.00 | 0.2814 | 14.32 | 599.39 | 131.91 | 1.74 | 9.820 | 9.984 | 8.232 |

Individual Pharmacokinetic Parameters - Colchicine with Grapefruit Juice (All subjects)

| | | | | | | | | | | | | Weight- | | | |
|---|---|---|---|---|---|---|---|---|---|---|---|---|---|---|---|
| | | | | | | | | | | | | adjusted | | | |
| | | | | AUC₀₋ₜ | AUC_{0-∞} | | | | | | | CL/F | | | |
| Sub- | Study | Gen- | Treat- | (pg-hr/ | (pg-hr/ | Cₘₐₓ | Tₘₐₓ | Kel | t_{1/2} | Vₐᵣₑₐ/F | CL/F | (L/hr/ | Ln-Transformed | | |
| ject | Day | der | ment | mL) | mL) | (pg/mL) | (hr) | (1/hr) | (hr) | (L) | (L/hr) | kg) | AUC₀₋ₜ | AUC_{0-∞} | Cₘₐₓ |
| 1 | Day 18 | Female | A + B | 11579.75 | 13449.06 | 2991 | 1.50 | 0.1658 | 4.18 | 269.02 | 44.61 | 0.75 | 9.357 | 9.507 | 8.003 |
| 2 | Day 18 | Male | A + B | 7182.30 | 8917.15 | 1565 | 1.50 | 0.1207 | 5.74 | 557.46 | 67.29 | 0.68 | 8.879 | 9.096 | 7.356 |
| 3 | Day 18 | Female | A + B | 9868.35 | 11017.09 | 2094 | 2.00 | 0.2141 | 3.24 | 254.42 | 54.46 | 0.73 | 9.197 | 9.307 | 7.647 |
| 5 | Day 18 | Male | A + B | 9423.95 | 11182.03 | 2530 | 1.50 | 0.1369 | 5.06 | 391.92 | 53.66 | 0.60 | 9.151 | 9.322 | 7.836 |
| 6 | Day 18 | Male | A + B | 7231.95 | 9341.00 | 1698 | 1.50 | 0.1191 | 5.82 | 539.29 | 64.23 | 0.70 | 8.886 | 9.142 | 7.437 |
| 7 | Day 18 | Female | A + B | 8614.65 | 10702.57 | 2135 | 1.00 | 0.1171 | 5.92 | 478.74 | 56.06 | 0.83 | 9.061 | 9.278 | 7.666 |
| 8 | Day 18 | Male | A + B | 8785.10 | 10464.37 | 1751 | 1.50 | 0.1577 | 4.39 | 363.48 | 57.34 | 0.71 | 9.081 | 9.256 | 7.468 |
| 9 | Day 18 | Male | A + B | 8623.55 | 10278.84 | 2292 | 1.50 | 0.1370 | 5.06 | 426.03 | 58.37 | 0.74 | 9.062 | 9.238 | 7.737 |
| 10 | Day 18 | Male | A + B | 5740.85 | 6992.12 | 1783 | 1.00 | 0.2172 | 3.19 | 395.04 | 85.81 | 1.04 | 8.655 | 8.853 | 7.486 |
| 11 | Day 18 | Male | A + B | 8524.00 | 9984.83 | 1814 | 1.50 | 0.1669 | 4.15 | 360.06 | 60.09 | 0.61 | 9.051 | 9.209 | 7.503 |
| 12 | Day 18 | Male | A + B | 14329.00 | 19676.87 | 2143 | 1.50 | 0.0449 | 15.44 | 679.18 | 30.49 | 0.30 | 9.570 | 9.887 | 7.670 |
| 13 | Day 18 | Male | A + B | 8954.90 | 10403.74 | 1786 | 1.00 | 0.1865 | 3.72 | 309.24 | 57.67 | 0.65 | 9.100 | 9.250 | 7.488 |
| 14 | Day 18 | Male | A + B | 7754.50 | 9194.41 | 1817 | 2.00 | 0.1597 | 4.34 | 408.54 | 65.26 | 0.75 | 8.956 | 9.126 | 7.505 |
| 15 | Day 18 | Female | A + B | 12948.30 | 16163.97 | 2190 | 1.00 | 0.0667 | 10.39 | 556.22 | 37.12 | 0.55 | 9.469 | 9.691 | 7.692 |
| 16 | Day 18 | Male | A + B | 5632.63 | 7194.22 | 1848 | 1.00 | 0.1713 | 4.05 | 486.87 | 83.40 | 1.18 | 8.636 | 8.881 | 7.522 |
| 17 | Day 18 | Male | A + B | 5993.05 | 7288.82 | 1554 | 1.50 | 0.2388 | 2.90 | 344.75 | 82.32 | 1.04 | 8.698 | 8.894 | 7.349 |
| 18 | Day 18 | Female | A + B | 15918.25 | 22023.85 | 1888 | 3.00 | 0.0477 | 14.53 | 571.01 | 27.24 | 0.36 | 9.675 | 10.000 | 7.543 |
| 19 | Day 18 | Male | A + B | 10440.30 | 11956.83 | 2522 | 2.00 | 0.1817 | 3.81 | 276.13 | 50.18 | 0.60 | 9.253 | 9.389 | 7.833 |
| 20 | Day 18 | Male | A + B | 5166.95 | 6675.90 | 1611 | 1.00 | 0.1849 | 3.75 | 486.09 | 89.88 | 1.06 | 8.550 | 8.806 | 7.385 |
| 21 | Day 18 | Male | A + B | 8590.10 | 10054.98 | 2145 | 2.00 | 0.1634 | 4.24 | 365.28 | 59.67 | 0.56 | 9.058 | 9.216 | 7.671 |
| 22 | Day 18 | Male | A + B | 3988.98 | 4975.96 | 1236 | 1.00 | 0.2095 | 3.31 | 575.48 | 120.58 | 1.59 | 8.291 | 8.512 | 7.120 |

| | | | | | | | | | | | | |
|---|---|---|---|---|---|---|---|---|---|---|---|---|
| N | 21 | 21 | 21 | 21 | 21 | 21 | 21 | 21 | 21 | 21 | 21 | 21 |
| MEAN | 8823.40 | 10854.22 | 1971.10 | 1.50 | 0.1527 | 5.58 | 433.06 | 62.18 | 0.76 | 9.030 | 9.231 | 7.567 |
| STDEV | 3003.15 | 4140.12 | 398.80 | 0.50 | 0.05 | 3.51 | 117.21 | 21.42 | 0.29 | 0.34 | 0.35 | 0.20 |
| % CV | 34.04 | 38.14 | 20.23 | 33.33 | 34.78 | 62.79 | 27.06 | 34.45 | 37.88 | 3.79 | 3.80 | 2.63 |
| MEDIAN | 8614.65 | 10278.84 | 1848 | 1.50 | 0.1634 | 4.24 | 408.54 | 58.37 | 0.71 | 9.061 | 9.238 | 7.522 |
| MIN | 3988.98 | 4975.96 | 1236 | 1.00 | 0.0449 | 2.90 | 254.42 | 27.24 | 0.30 | 8.291 | 8.512 | 7.120 |
| MAX | 15918.25 | 22023.85 | 2991 | 3.00 | 0.2388 | 15.44 | 679.18 | 120.58 | 1.59 | 9.675 | 10.000 | 8.003 |

Recitation of ranges of values herein are merely intended to serve as a shorthand method of referring individually to each separate value falling within the range, unless otherwise indicated herein, and each separate value is incorporated into the specification as if it were individually recited herein. The endpoints of all ranges directed to the same component or property are inclusive and independently combinable.

All methods described herein can be performed in a suitable order unless otherwise indicated or clearly contradicted by context. The use of any and all examples, or exemplary language (e.g., “such as”) herein is intended to better illuminate the disclosure and is non-limiting unless otherwise specified. No language in the specification should be construed as indicating that any non-claimed element as essential to the practice of the claimed embodiments. Unless defined otherwise, technical and scientific terms used herein have the same meaning as is commonly understood by one of skill in the art to which this disclosure belongs. The terms wt %, weight percent, percent by weight, etc. are equivalent and interchangeable. The modifier “about” used in connection with a quantity is inclusive of the stated value and has the meaning dictated by the context (e.g., includes the degree of error associated with measurement of the particular quantity).

Embodiments are described herein, including the best modes known to the inventors. Variations of such embodiments will become apparent to those of ordinary skill in the art upon reading the foregoing description. The skilled artisan is expected to employ such variations as appropriate, and the disclosed methods are expected to be practiced otherwise than as specifically described herein. Accordingly, all modifications and equivalents of the subject matter recited in the claims appended hereto are included to the extent permitted by applicable law. Moreover, any combination of the above-described elements in all possible variations thereof is encompassed unless otherwise indicated herein or otherwise clearly contradicted by context.

I claim:

1. A method of administering colchicine to a patient in need thereof, comprising
administering a daily dosage amount of colchicine orally to a patient consuming a daily amount of grapefruit juice that does not exceed about 16 fluid ounces.

2. The method of claim 1 wherein the colchicine is administered to the patient to prevent gout flares.

3. The method of claim 2, wherein the daily dosage amount of colchicine is about 0.6 mg to about 1.2 mg.

4. The method of claim 1 wherein the colchicine is administered to the patient to treat an acute gout flare.

5. The method of claim 4, wherein the daily dosage amount of colchicine is about 1.2 mg followed by about 0.6 mg after about an hour.

6. The method of claim 4, wherein the daily dosage amount of colchicine is about 1.8 to about 4.8 mg.

7. The method of claim 1 wherein the colchicine is administered to the patient to treat familial Mediterranean fever.

8. The method of claim 7, wherein the daily dosage amount of colchicine for an adult patient is about 1 to about 3 mg.

9. The method of claim 7, wherein the daily dosage amount of colchicine for a child patient is about 0.25 mg to about 3 mg.

10. The method of claim 1, further comprising
monitoring the patient for colchicine toxicity.

11. The method of claim 1, wherein after administration of a 0.6 mg colchicine oral dosage form to human subjects on the fourth day of twice daily consumption of 8 fluid ounces of grapefruit juice, the logarithmic transformed area under the curve of colchicine plasma concentration measured as a function of time from time 0 to time t (AUC₀₋ₜ) is within about 80% to about 125% of 8807 hr*ng/ml;
the logarithmic transformed area under the curve of colchicine plasma concentration measured as a function of time from time 0 to time infinity (AUC_{0-∞}) is within about 80% to about 125% of 10500 hr*ng/ml; or
the logarithmic transformed maximum plasma concentration (Cₘₐₓ) of colchicine is within about 80% to about 125% of 2074 ng/ml.

12. The method of claim 1, wherein after administration of a 0.6 mg colchicine oral dosage form to human subjects on the fourth day of twice daily consumption of 8 fluid ounces of grapefruit juice, the colchicine oral dosage form is bioequivalent to the colchicine oral dosage form administered without consumption of grapefruit juice.

13. The method of claim 12, wherein the upper and lower limits of the 90% confidence interval of a ratio of a geometric mean of logarithmic transformed area under the curve of colchicine plasma concentration measured as a function of time from time 0 to time infinity (AUC_{0-∞}) of the 0.6 mg colchicine oral dosage form administered alone to that administered on the fourth day of twice daily consumption of 240 mL of grapefruit juice are about 0.80 to about 1.25.

14. The method of claim 12, wherein the upper and lower limits of the 90% confidence interval of a ratio of a geometric mean of logarithmic transformed area under the curve of colchicine plasma concentration measured as a function of time from time 0 to time t (AUC₀₋ₜ) of the 0.6 mg colchicine oral dosage form administered alone to that administered on the fourth day of twice daily dosing of 240 mL of grapefruit juice are about 0.80 to about 1.25.

15. The method of claim 12, wherein the upper and lower limits of the 90% confidence interval of a ratio of a geometric mean of logarithmic transformed maximum plasma colchicine concentration (Cₘₐₓ) of 0.6 mg colchicine administered alone to colchicine administered on the fourth day of twice daily dosing of 240 mL of grapefruit juice is about 0.7 to about 1.43.
